(19) United States
(12) Patent Application Publication
(10) Pub. No.: US 2005/0009168 A1
(43) Pub. Date: Jan. 13, 2005

(54) Methods and apparatus for Adeno associated virus purification

(76) Inventors: Joan Marie Robbins, San Diego, CA (US); Marylou G. Gibson, Carlsbad, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 10/843,718

(22) Filed: May 11, 2004

Related U.S. Application Data
(60) Provisional application No. 60/469,974, filed on May 12, 2003.

(51) Int. Cl. C12N 7/02; C12M 1/34

(52) U.S. Cl. 435/239; 435/287.1

(57) ABSTRACT

The present invention provides an apparatus for the purification of Adeno-associated virus (AAV) and methods of use including an anion exchange filter unit and a cation exchange capture unit. At one end, the cation exchange capture unit reversibly engages the anion exchange filter unit and when engaged is in fluid communication. The cation exchange capture unit may, at the opposing end, reversibly engage a syringe or apparatus able to provide negative pressure to draw a fluid containing AAV through the anion exchange filter unit then cation exchange capture unit where the AAV is captured. The anion exchange filter unit is disengaged and the purified AAV is eluted from the cation exchange capture unit.

### CROSS REFERENCE TO RELATED APPLICATIONS

The present application claims benefit of priority to U.S. Provisional Application Ser. No. 60/469,974, entitled, “Methods and Apparatus for Adeno Associated Virus Purification” filed on May 12, 2003, and is herein incorporated by reference in its entirety.

### TECHNICAL FIELD

The present invention relates to the purification of Adeno-associated virus and more specifically to an apparatus for purifying Adeno-associated virus including an anion exchange filter unit able to reversibly engage a cation exchange capture unit.

### BACKGROUND

Adeno-associated virus (AAV) has unique features, which can be utilized to deliver genes for gene therapy. AAV is capable of infecting a wide variety of cell types, but has not been implicated in causing human disease. Thus it is a useful vector to carry therapeutic genes into human cells.

AAV particles are composed of an icosahedral capsid containing assemblies of the three CAP gene products, VP1, VP2 and VP3. Inside the capsid is the 4.6 kb single stranded DNA genome. AAV is a small virus when compared to most other viruses; it is a dependovirus, and replicates only in the presence of other specific larger DNA viruses, utilizing their replicative functions. AAV normally replicates only in human cells that are also co-infected with a helper virus such as adenovirus, herpesvirus or poxvirus. It relies on its helper virus to encode several viral genes necessary for self-replication. Dependoviruses need to carry little to ensure their own survival; they encode only minimal structural genes and genes directing DNA replication and encapsidation. Its REP gene, which is involved in AAV specific DNA duplication can be replaced in the AAV genome with the investigator's gene of interest, generating virus particles that cannot self replicate. The defect in the structural gene and absence of the helper virus can be replaced or complemented with structural genes in trans, either on a separate helper plasmid DNA or expressed by a expressing cell line.

AAV can be generated in tissue culture at high concentrations, and technologies allow it to be made in the absence of other helper viruses. For example, U.S. Pat. Ser. Nos. 6,632,670 and 6,566,118 describe methods of producing high titer recombinant AAV in the absence of infectious helper virus by using cloned AAV genes as well as cloned gene of interest to transfect producer cell lines.

Current methods for the purification of Adeno-associated virus involve the use of density gradient centrifugation utilizing cesium chloride or other density gradient media such as metronidazole. Alternatively, it has been reported that column chromatography resins, specifically the ion exchange resins or resins modified with Heparin sulfate can be utilized to purify AAV. These descriptions require chromatographic fractionation procedures wherein crude AAV material is added to a chromatography resin and then eluted with a range of buffers with various characteristics such as ionic strength or pH. Then a select range within that elution may be applied to a second column chromatography resin and after washing and elution procedures, purified material may be eluted. Thus the purification of AAV using current technologies may be time consuming and complex.

### SUMMARY OF THE INVENTION

The present invention addresses the shortcomings in current techniques for the purification of Adeno-associated virus (AAV). Accordingly, one aspect of the present invention provides an apparatus for the purification of Adeno-associated virus including an anion exchange filter unit able to reversibly engage a cation exchange capture unit. When engaged, the anion exchange filter unit is in fluid communication with the cation exchange capture unit. Optionally, the cation exchange unit is able to reversibly engage a structure, such as a syringe or other vacuum source, which is able to draw fluid through the anion exchange unit and the cation exchange capture unit.

In another aspect of the present invention, an apparatus for the purification of Adeno-associated virus is provided including a connecting structure positioned between and fluidly connecting an anion exchange filter unit and a cation exchange capture unit. The connecting structure may be provided in a variety of configurations such as flexible or rigid tubing. The connecting structure reversibly connects the anion exchange filter unit to the cation exchange filter unit allowing disconnection of the units.

In another aspect of the present invention, a kit for the purification of Adeno-associated virus is provided including an anion exchange filter unit, a cation exchange capture unit and one or more buffers such as a dilution buffer, a wash buffer or an elution buffer.

In another aspect of the present invention, a method of purifying Adeno-associated virus is provided including providing an engaged apparatus for purifying Adeno-associated virus, drawing an aqueous solution including an Adeno-associated virus through the anion filter exchange unit then through the cation exchange filter unit, where the Adeno-associated virus is captured, disengaging the anion exchange filter unit, and eluting the Adeno-associated virus from the cation exchange filter unit.

### BRIEF DESCRIPTION OF THE DRAWINGS

FIG. 1 depicts an apparatus 10 of the present invention including an anion exchange filter unit 11 engaged to a cation exchange capture unit 12. The cation capture unit 12 is shown engaged to a syringe 13 at the opposing end.

FIG. 2 depicts an elution configuration of the present invention 10. The cation exchange capture unit 12 is shown engaged to a syringe 13.

FIGS. 3A and 3B are a table depicting Fluorescent Activated Cell Sorter (FACS) Data of HT1080 cells treated with various AAV-Green Fluorescent Protein samples from AAV purification runs.

FIG. 4 is a Western Blot stained with Comassie Blue depicting results of a preparation using a variety of disclosed configurations and buffers.

### DETAILED DESCRIPTION OF THE INVENTION

The present invention provides a device and method for the purification of Adeno-associated virus (AAV). The present invention permits both the lysed cell extract and the supernatant to be utilized in AAV purification. Up to about 50% of AAV particles can be released from the producer cells during AAV production, and with popular centrifugation based purification methods, this supernatant is routinely discarded. With the methodology described herein, the virus in both the lysed cell extract and in the supernatant may be purified. Additional benefits of the present invention include increased speed and simplicity employed to purify AAV.

The present invention includes a purification device 10, which may be employed by a researcher or biomanufacturer for production of AAV. The disclosed apparatus 10 combines multiple purification functions in one action and is not affected by the changes in flow rate of fluid containing AAV. For example, the apparatus 10 may be used with a variety of flow rates such as but not limited to from about 1 mL per minute to about 60 mL per minute. Although results may vary, the present invention will typically purify from about 1×10e5 through 1×10e13 virus particles. The result is the ability to rapidly, efficiently and easily purify AAV or similarly charged viruses without the use of a centrifuge.

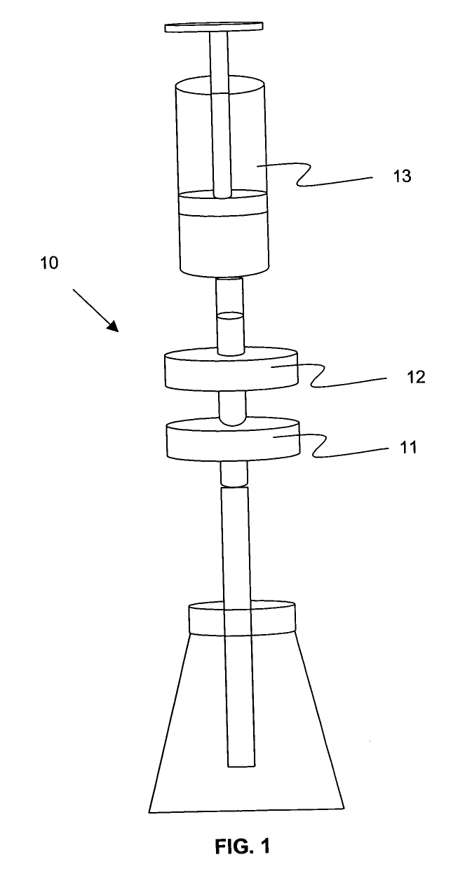

Referring to FIG. 1, the present invention provides an apparatus 10 for the purification of Adeno-associated virus including an anion exchange filter unit 11 and a cation exchange capture unit 12. At one end, the cation exchange capture unit 12 reversibly engages the anion exchange filter unit 11 and when engaged is in fluid communication. The cation exchange capture unit 12 may, at the opposing end, reversibly engage a syringe 13 or apparatus able to provide negative pressure through the cation exchange capture unit 12 and anion exchange filter unit 11, which draws a fluid through the device 10.

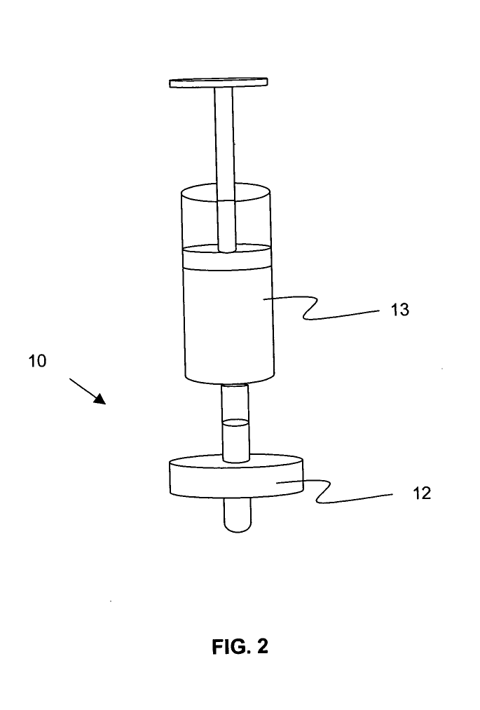

The present invention utilizes an anion exchange filter unit 11 to remove contaminants and a cation exchange capture unit 12 to capture the AAV. In one embodiment a syringe 13 engaged to the cation exchange capture unit 12 is drawn. The negative pressure pulls a fluid through the anion exchange filter unit 11, through the cation exchange capture unit 12 and into the syringe. The syringe 13 and anion exchange filter unit 11 are disengaged from the cation exchange capture unit 12. A syringe 13 containing a wash buffer may be engaged to the cation exchange capture unit 12 and pressed to exert positive pressure or alternatively attached to the filter and used to exert negative pressure to pull wash buffer through the cation exchange capture unit 12 to wash potential contaminants from the cation exchange capture unit 12. As demonstrated in FIG. 2, the AAV is eluted by administering an elution buffer to the cation exchange capture unit 12. AAV can be eluted from the unit 12 in either direction.

In another embodiment of the present application, a structure such as syringe 13 or a pump provides positive pressure flow to the anion exchange filter unit 11. The fluid passes from the anion exchange filter unit 11 through the cation exchange capture unit 12 where the AAV or similarly charged virus is captured. The fluid continues to flow through the device 10 and is discarded. The anion exchange filter unit 11 is disengaged and discarded and the cation exchange filter unit is washed, such as by providing a wash buffer. The AAV is eluted using an elution buffer.

The principle employed for purification is based on the charge of the virus, and not on specific ligands on the virus capsid. The present apparatus 10 and method can be utilized to purify AAV serotypes 1, 2, 3, 4, 5 and 6. This is an improvement over ligand-based chromatographic purification methods utilizing heparin since Heparin Sepharose purification chromatography columns will not efficiently purify AAV types 1,4,5 or 6.

The present apparatus 10 and method may also be used for the purification for other parvoviruses having a charge similar to AAV, including other defective and replication competent parvoviruses. For example, the present invention 10 may be utilized to purify viruses such as but not limited to B19, minute virus of mice, feline parvovirus, canine parvovirus, Aleutian parvovirus, insect parvoviruses and other similarly charged viruses.

The fluid containing the AAV to be purified should be at an appropriate pH for proper functioning of the anion exchange filter unit 11 and the cation exchange capture unit 12. The pH may be from about 6.5 to about 7.5, preferably about 7.1. The pH of the AAV containing fluid may be adjusted or stabilized using a dilution buffer prior to its being applied to the purification unit.

The fluid or supernatant may also be clarified prior to utilizing the present device 10. Particulates, cellular debris or other contaminants may be removed using techniques such as centrifugation or filtration, such as by using a 0.45 micron filter. The fluid or supernatant may be treated with DNAse to remove extra-capsid chromosomal and input plasmid DNA prior to applying the solution to the AAV purification unit 10.

The anion exchange filter unit 11 is able to attract and bind undesirable contaminants such as cellular proteins, trace cellular and plasmid DNAs, cellular contaiminants and contaminating adenovirus when used in the preparation of AAV. Since the production of AAV requires Adenovirus helper functions, these can be supplied by live adenovirus or more recently by Adenovirus functions encoded by input plasmid DNA. The anion exchange filter unit 11 does not significantly capture the AAV. The anion exchange filter unit 11 can be constructed from a variety of membranes available in the biotechnology arts or chemical arts such as but not limited to Sartobind Q (Sartorius AG, Goettingen, Germany) and Pall Mustang Q (Pall Corp, Ann Arbor, Mich.). Alternatively the anion exchange unit 11 may be constructed from one or more of a variety of matrices known in the biotechnology or chemical arts such as but not limited to DEAE media (Amersham, Piscataway, N.J.), Q media (Amersham, Piscataway, N.J.), Source Q (Amersham, Piscataway, N.J.), Monobead Q (Biosepra, Marlborough, Mass.), Sepharose Big Bead Q (Amersham, Piscataway, N.J.) and Unosphere (BioRad, Hercules, Calif.).

The disclosed membranes or matrices may be provided in a variety of configurations that allow a fluid to pass through the anion exchange filter unit 11. For example, a membrane may be provided in a disk-like configuration encased in an injection molded polypropylene or polystyrene housing. The surface area of the membrane may be provided in a variety of sizes. For example the membrane may be from about 3 to about 100 square centimeters or up to several square meters. Alternatively, a matrix may be provided in a cassette-like configuration where the matrix is encased within an injection molded polypropylene or polystyrene cassette-like housing. The cassette may be provided in a variety of non-limiting sizes. For example, a cassette may be provided to house from about 1 mL to about 100 mL or a 1 L or more. The size of a membrane or cassette may depend on the volume of contaminant to bind or filter.

The cation exchange capture unit 12 captures the AAV. The cation exchange capture unit 12 can be constructed from a variety of membranes available in the biotechnology arts or chemical arts such as but not limited to Sartobind S membrane (Sartorius AG, Goettingen, Germany) or a Pall Mustang S membrane (Pall Corp, Ann Arbor, Mich.). Alternatively the cation exchange capture unit 12 may be constructed from a variety of matrices known in the biotechnology or chemical arts such as but not limited to S media (Amersham, Piscataway, N.J.), CM media (Amersham, Piscataway, N.J.), Source S (Amersham, Piscataway, N.J.), a Monobead (Biosepra, Marlborough, Mass.), a Big Bead (Amersham, Piscataway, N.J.), and a Unosphere matrix (BioRad, Hercules, Calif.).

The disclosed cation exchange membranes or matrices may be provided in a variety of configurations that allow a fluid to pass through the device 12. For example, a membrane may be provided in a disk-like configuration encased in an injection molded polypropylene or polystyrene housing. The surface area of the membrane may be provided in a variety of sizes. For example the membrane may be from about 3 to about 100 square centimeters or up to several square meters. Alternatively, a matrix may be provided in a cassette-like configuration where the matrix is encased within an injection molded polypropylene or polystyrene housing. The cassette may be provided in a variety of non-limiting sizes. For example, a cassette may be provided to house from about 1 mL to about 100 mL or 1 L or more. The size of a membrane or cassette may depend on the volume of virus to purify.

The anion exchange filter unit 11 and the cation exchange capture unit 12 are engaged such that a fluid may pass through each unit with the anion filter unit 11 preceding the cation exchange capture unit 12. The units are removable to permit discarding of the anion exchange unit 11. Thus a variety of configurations may allow the reversible engagement of these units. For example, the cation exchange capture unit 12 and the anion exchange filter unit 11 may reversibly engage using surfaces which are complementary and thus may interlock, snap, clasp twist-lock and the like. In one embodiment the apparatus 10 engages using luer locks.

In another embodiment a connecting structure connects the anion exchange unit 11 to the cation capture unit 12. A connecting structure allows fluid communication between the units but does not require direct engagement. For example, a connecting structure such as rigid or flexible tubing may be placed between the anion exchange filter unit 11 and the cation exchange capture unit 12. The connecting structure may connect to either unit using a variety of techniques known in the biotechnology or mechanical arts such as but not limited to adapters, barbed fittings, clasps and the like.

In another embodiment the anion exchange filter unit 11 and cation exchange capture unit 12 are provided in an engaged configuration. The user draws a fluid through the anion exchange filter unit 11 and cation exchange capture unit 12. The anion exchange filter unit 11 is detached or disengaged such as by snapping the device 10 along an etched portion or region.

The present invention may also be provided in a kit format. For example, a kit may include an anion exchange filter unit 11, a cation exchange capture unit 12 and one or more buffers. The buffers may include a dilution buffer for adjusting or equilibrating the pH of a solution containing AAV, a wash buffer for washing the cation exchange capture unit 12 or an elution buffer for eluting the AAV from the cation exchange capture unit 12.

As non-limiting examples, a dilution buffer may include 400 mM Tris pH 6.7 ±0.3 in, a wash buffer may include 115 mM NaCl, 20 mM Tris pH 7.5 and an elution buffer may include 400 mM NaCl, 20 mM Tris pH7.5.

The present invention also provides a method of purifying AAV from a solution containing AAV. The method includes providing an engaged apparatus 10 as substantially disclosed, drawing an aqueous solution containing AAV through the anion exchange filter, capturing the AAV in the cation exchange capture unit 12, disengaging the anion exchange filter unit 11 and eluting the AAV from the cation exchange capture unit 12. Optionally, the disclosed method includes washing the cation exchange capture unit 12 prior to elution.

### EXAMPLES

### Example 1

### Preparation of AAV from HEK293 Cells

HEK293 cells or their variants can be grown in tissue culture treated flasks. For the production of AAV, cells should be at a relatively early passage level. They should be kept on a regular passage program. Cells should not remain confluent for more than a few days. Cell that have remained confluent and unpassed for a more than several days can be passed at least one time at a low seeding density to reset the cells into an active growing state.

Cells may be seeded into the tissue culture flask at approximately 4×10⁴ cells per cm². Recommended media: DMEM, high glucose with 4 mM glutamine and 10% Fetal Calf Serum. This media can be purchased through a variety of vendors such as Life Sciences. JRH, Mediatech, or Irvine Scientific. The cell monolayer may become nearly confluent within approximately 2 to 4 days. Cell cultures at optimal cell density may be transfected with the plasmid or plasmids which will provide the necessary genes for the production of AAV. These genes may be on one large plasmid or on several different plasmids, but the critical genes for AAV production include the AAV rep gene, AAV cap gene, the Adenovirus helper gene or other viral helper gene and the expressed gene of interest flanked by the AAV Iterated Terminal Repeats. After transfection, harvest the cultures within 2 to 5 days by gently shaking or pipetting the cells off of the culture dish.

At harvest, pool all the cell lysate and media into one capped vessel and freeze and thaw at least three times. The cell debris may be centrifuged, pelletted and discarded at approximately 2500 g for 20 to 30 minutes, and the resulting clarified supernatant is treated with DNase I, Benzonase or an equivalent for 30 minutes to 1 hour at 37° C. Clarified supernatant may then be filtered through a glass fiber filter and then through a 0.45 or 0.2 micron cellulose acetate or PDGF membrane. The resulting filtrate is then adjusted with one tenth volume of 200 mM Tris, pH 6.8+/−0.3 pH units. pH adjustment is critical for stabilization of the pH of the tissue culture fluids and may be achieved with a variety of volumes of buffers.

### Example 2

### Purifying/Concentrating AAV from the Lysed Cells and Supernatant

The anion filter exchange unit 11 and cation capture unit 12 are engaged so that the supernatant flows across the anion filter exchange unit 11 then the cation exchange capture unit 12. The supernatant is passed through the apparatus 10 at approximately 10 to 20 mL per minute. When the entire supematant has been passed over the filter assembly, the anionic exchange filter unit 11 is disengaged such as by twisting the easily detachable luer lock and the filter 11 is discarded. The remaining cationic exchange capture unit 12 is washed by passing at least 40 mL of 115 mM NaCl, 20 mM Tris pH 7.5 over the filter at the rate of 10-20 mL per minute. AAV is eluted from the filter with 2 to 3 mL of 400 mM NaCl, 20 mM Tris pH 7.5.

### Example 3

### Comparison of Transducing Ability of Purified AAV on Human HT1080 Cells

In order to show that the orientation of the anionic and cationic filters is critical to the purification of AAV from infected cells and supernatants, 50 mL of crude AAV-GFP supernatants were applied to the units in both the proscribed orientations of anionic then cationic or the reverse, cationic then anionic. In addition, the crude supernatants were pH adjusted to either pH 6.8 or 7.1 and then applied to the purification units in their anionic, cationic orientation. The two units were then disassociated and the second unit was washed with 40 mL of 115 mM NaCl, pH 7.4. The AAV particles present were eluted with 400 mM NaCl, 20 mM tris, pH 7.5 into 1.5 ml of elution buffer. The purified AAV samples were then used to transduce HT1080 cells. The result of transduction with AAV-GFP will be cells that intracellularly fluoresce green at the appropriate wavelength. Samples of 0.100 and 0.025 mL of purified AAV-GFP purified in the various methods described above were used to transducer 2×10e5 HT1080 cells. 48 hours after transduction, the cells were dissociated from the culture dish and the percent green cells were determined by FACS analysis.

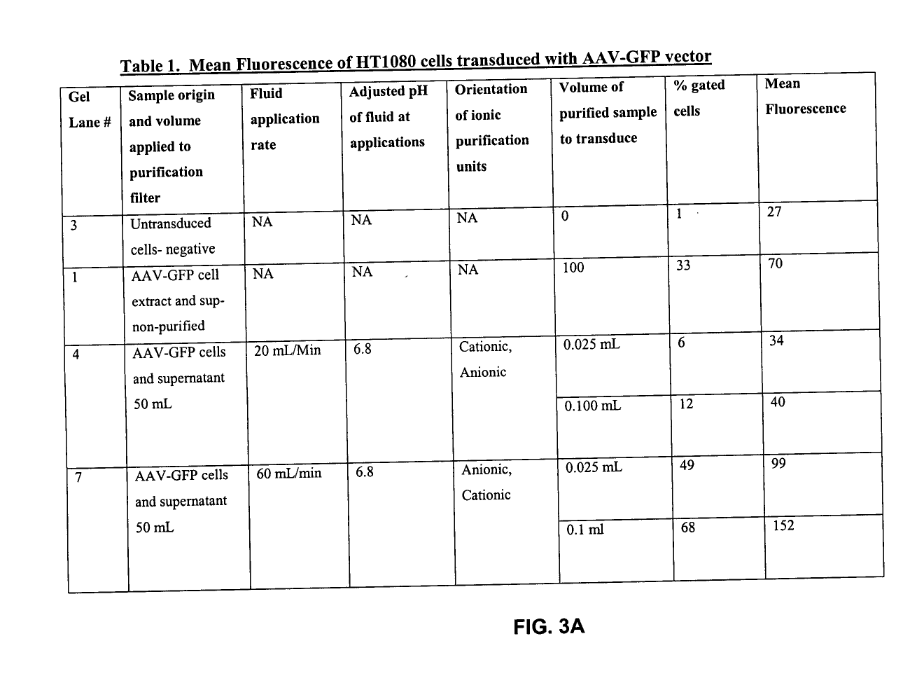

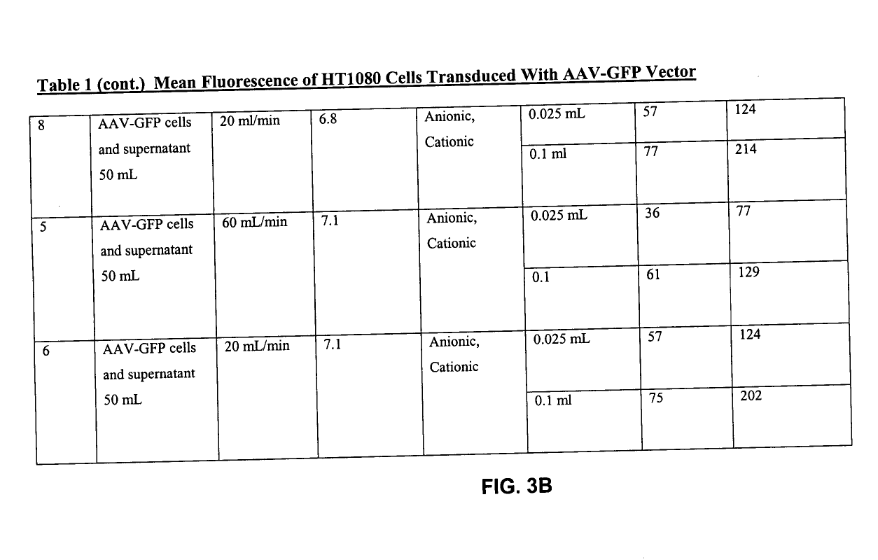

Referring to FIGS. 3A and 3B, Table 1 depicts Fluorescent Activated Cell Sorter (FACS) Data of HT1080 cells treated with various AAV-Green Fluorescent Protein samples from AAV purification runs. The virus has effectively been concentrated by [10] fold. This experiment shows that variations in the pH at which the AAV samples are stabilized prior to their application to the AAV purification unit and the speed at which the samples are applied to the unit do not effect the quality of purified AAV-Green fluorescent protein which can be obtained. The intensity of the fluorescing cells in the purified samples is similar regardless of the speed of virus supernantant application. However, the orientation of the filters is important as the level of fluorescence is reduced in sample 4 (cation first followed by anion filter). In addition, it is shown that both adjusted pHs of 6.8 and 7.1 are adequate for the purification of AAV.

### Example 4

### Purity of AAV Vector Preparations

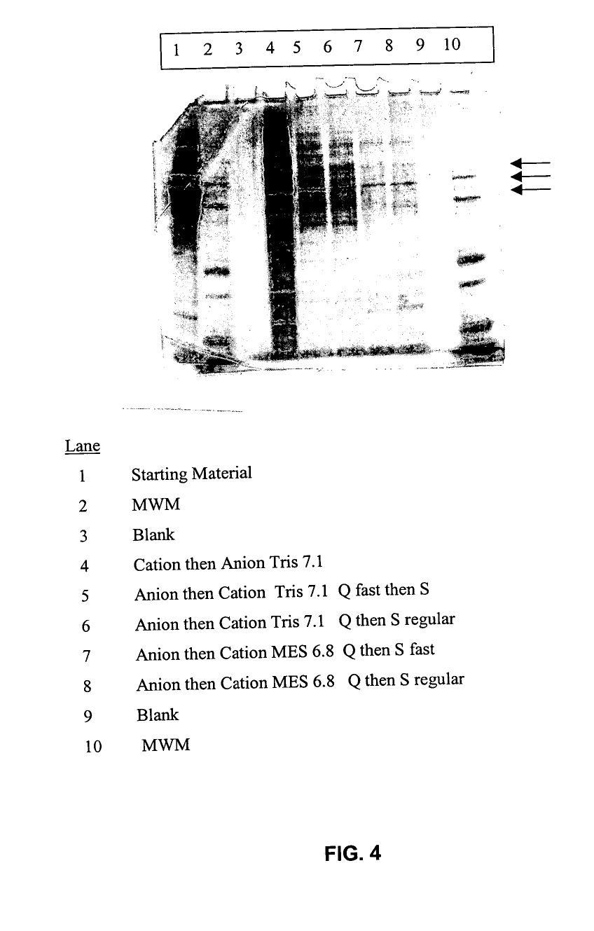

In order to examine the purity of AAV vector prepared as in example 2, samples are subjected to acrylamide gel electrophoresis and subsequent staining with Comassie Blue stain. Referring to FIG. 4, Contaminants show as multiple bands in the gel. The approximate location of AAV capsid proteins VP1, VP2 and VP3 are shown by arrows. The heavily contaminated starting material is shown in lane 1. Lane 4 shows extensive contamination with cellular proteins, whereas lanes 5-8 are relatively clean. This demonstrates that the orientation of the anionic and cationic filters is critical in order to achieve optimal purification.

1. An apparatus for the purification of Adeno-associated virus comprising:
an anion exchange filter unit;
a cation exchange capture unit able to reversibly engage said anion exchange filter unit; and
wherein when said anion exchange filter unit is in fluid communication with said cation filter capture unit when engaged.

2. The apparatus for the purification of Adeno-associated virus according to claim 1, wherein said anion exchange filter unit comprises a Sartobind Q membrane or a Pall Mustang Q membrane.

3. The apparatus for the purification of Adeno-associated virus according to claim 1, wherein said anion exchange filter unit comprises a matrix selected from the group consisting of DEAE media, Q media, Source Q, Monobead Q, Sepharose Big Bead Q and Unosphere.

4. The apparatus for the purification of Adeno-associated virus according to claim 1, wherein said cation exchange capture unit comprises a Sartobind S membrane or a Pall Mustang S membrane.

5. The apparatus for the purification of Adeno-associated virus according to claim 1, wherein said cation exchange capture unit comprises a matrix selected from the group consisting of S media, CM media, Source S, a Monobead, a Big Bead, and a Unosphere matrix.

6. The apparatus for the purification of Adeno-associated virus according to claim 1, wherein said anion exchange filter unit and said cation exchange capture unit comprise complementary engaging surfaces.

7. The apparatus for the purification of Adeno-associated virus according to claim 6, wherein said complementary engaging surfaces comprise luer lock structures.

8. The apparatus for the purification of Adeno-associated virus according to claim 1, further comprising a syringe able to be reversibly engaged to said cation exchange capture unit at an end opposite said anion exchange filter unit.

9. The apparatus for the purification of Adeno-associated virus according to claim 1, further comprising a vacuum device able to be reversibly engaged to said cation exchange capture unit at an end opposite said anion exchange filter unit.

10. An apparatus for the purification of Adeno-associated virus comprising:
an anion exchange filter unit;
a cation exchange filter unit;
a connecting structure able to reversibly connect said anion exchange filter unit to said cation exchange filter unit; and
wherein said anion exchange filter unit is in fluid communication with said cation exchange capture unit when connected.

11. A kit for the for the purification of Adeno-associated virus comprising:
a) an apparatus for the purification of Adeno-associated virus according to claim 1;
b) a dilution buffer;
c) a wash buffer; and
d) an elution buffer.

12. A method of purifying Adeno-associated virus comprising:
a) providing an engaged apparatus for the purification of Adeno-associated virus according to claim 1;
b) drawing an aqueous solution comprising an Adeno-associated virus through said anion filter exchange unit;
c) capturing said Adeno-associated virus within said cation exchange capture unit;
d) disengaging said anion exchange filter unit; and
e) eluting said Adeno-associated virus from said cation exchange filter unit.
